(19) United States
(12) Patent Application Publication
(10) Pub. No.: US 2005/0118283 A1
(43) Pub. Date: Jun. 2, 2005

(54) Skincare compositions and methods

(76) Inventors: Ruth Rebecca Calverley, Nottingham (GB); Joy Diane Thomas, Nottingham (GB); Lloyd George Hamilton, Nottingham (GB)

Correspondence Address: ROTHWELL, FIGG, ERNST & MANBECK, P.C., 1425 K STREET, N.W., SUITE 800, WASHINGTON, DC, 20005, US

(21) Appl. No.: 10/510,764

(22) PCT Filed: Apr. 9, 2003
(86) PCT No.: PCT/GB03/01523

(30) Foreign Application Priority Data
Apr. 9, 2002 (GB) 0208081.0

(51) Int. Cl. A61K 35/78; A61K 7/06; A61K 31/385; A61K 31/355; A61K 31/375; A61K 31/015

(52) U.S. Cl. 424/729; 424/765; 424/766; 424/769; 424/774; 424/778; 514/458; 514/440; 514/474; 424/74; 514/763

(57) ABSTRACT

A composition for the prevention or inhibition of free-radical-induced effects on the skin comprises (a) ascorbic acid, or a salt, ester, glucoside, glucosamine and/or other derivative thereof; (b) tocopherol, or an ester and/or other derivative thereof; and (c) at least one further anti-oxidant agent selected from the group consisting of: carotenoids; α-lipoic acid and salts thereof; green tea extract; hibiscus (Sudanese) tea extract; rooibus tea extract; embilica extract; rosehip extract; elderflower extract; and grape seed extract. The composition preferably comprises α-lipoic acid, or a salt thereof, and carotenoids, and is preferably administered orally, to provide systematic protection to the skin.

This invention relates to compositions providing enhanced protection for the skin against the adverse effects of free radicals, eg effects mediated by UV-radiation, or other sources of oxidative stress. In particular aspects, the invention provides compositions containing combinations of anti-oxidant agents effective in protecting the skin against damage due to free radicals, and orally-administered compositions providing systemic protection against such damage.

As we age, our skin undergoes changes such as becoming thinner, more easily damaged and less elastic. In addition, lifetime exposure to UV-A and UV-B radiation, together with other environmental factors that induce the formation of free radicals, such as pollution from trafficfumes, ozone, cigarette smoke etc, causes additional changes to the skin. These changes, including lines and wrinkling, actinic lentigines, dyspigmentation, rough skin, actinic telangiectasia and further loss of skin elastic function are due to direct UV-mediated damage to cells and indirectly mediated damage caused by the generation of free radicals in cells and tissues. This is generally termed photoageing and can account for up to 90% of the skin changes we associate with ageing.

The deleterious effects of UV radiation are generally believed to be due to the creation of free radicals. These highly reactive species may react with and damage DNA molecules in the skin (or elsewhere). Similar effects can also be attributed to radiation in the visible part of the spectrum.

It is known to use anti-oxidant compounds as free radical quenchers, thereby mitigating the effects of UV-mediated free radical formation.

There have now been devised compositions and methods involving combinations of free radical-scavenging agents which have been found to be particularly effective in protecting the skin against free radical-induced damage.

The invention utilises combinations of anti-oxidant agents including at least one, and more commonly more than one, anti-oxidant selected from the following list (List A):
carotenoids;
α-lipoic acid and salts thereof;
green tea extract;
hibiscus (Sudanese) tea extract;
rooibus (also known as “rooibos”) tea extract;
embilica extract;
rosehip extract;
elderflower extract; and
grape seed extract.

In a first aspect, the invention provides a composition for the prevention or inhibition of free radical-induced effects on the skin, which composition comprises
a) ascorbic acid, or a salt, ester, glucoside, glucosamine and/or other derivative thereof;
b) tocopherol, or an ester and/or other derivative thereof; and
c) at least one further anti-oxidant agent selected from List A.

In another aspect, the invention provides a method for the prevention or inhibition of free radical-induced effects on the skin, which method comprises the administration of a composition comprising
a) ascorbic acid, or a salt, ester, glucoside, glucosamine and/or other derivative thereof;
b) tocopherol, or an ester and/or other derivative thereof; and
c) at least one further anti-oxidant agent selected from List A.

The invention further provides the use of
a) ascorbic acid, or a salt, ester, glucoside, glucosamine and/or other derivative thereof;
b) tocopherol, or an ester and/or other derivative thereof; and
c) at least one further anti-oxidant agent selected from List A.
   in the manufacture of a composition for the prevention or inhibition of free radical-induced effects on the skin.

The skincare methods and compositions of the present invention are advantageous primarily in that they may protect the skin more effectively from the effects of UV-induced free radical formation than known skincare compositions. Therefore, the compositions and methods of the invention may be used to provide improved protection against damage to skin caused by exposure to factors such as sunlight, environmental and/or atmospheric pollution. The improved protection may be due to achievement of a greater degree of protection, a greater duration or protection and/or a more rapid onset of protection. The method of the invention may have therapeutic benefits, but its primary effect may be cosmetic in that it improves or prevents degradation of the appearance of the skin, eg due to the effects of exposure to external factors of the type that have been mentioned above, or due to ageing.

Some or all of the anti-oxidant agents used in the present invention may already be known to be effective as free radical quenchers and to prevent oxidative damage to the skin. However, the present invention discloses that combinations of these agents may have a greater efficacy than that expected. This has been demonstrated by in vitro and in vivo testing.

The combinations of anti-oxidant agents according to the invention may be administered by a variety of routes. The antioxidants may, for example, be formulated for topical administration to the skin. However, the combinations of antioxidants have been found to be particularly suitable for systemic administration, most commonly orally.

Thus, according to a further aspect of the invention there is provided a composition for the prevention or inhibition of free radical-induced effects on the skin, which composition is in a form suitable for oral administration and comprises
a) ascorbic acid, or a salt, ester, glucoside, glucosamine and/or other derivative thereof;
b) tocopherol, or an ester and/or other derivative thereof; and
c) at least one further anti-oxidant agent selected from List A.

In another aspect, the invention provides a method for the prevention or inhibition of free radical-induced effects on the skin, which method comprises the oral administration of a composition comprising
a) ascorbic acid, or a salt, ester, glucoside, glucosamine and/or other derivative thereof;
b) tocopherol, or an ester and/or other derivative thereof; and
c) at least one further anti-oxidant agent selected from List A.

The invention further provides the use
a) ascorbic acid, or a salt, ester, glucoside, glucosamine and/or other derivative thereof;
b) tocopherol, or an ester and/or other derivative thereof; and
c) at least one further anti-oxidant agent selected from List A
   in the manufacture of a composition for the prevention or inhibition of free radical-induced effects on the skin, the composition being in a form suitable for oral administration.

The composition according to the invention preferably comprises two or more anti-oxidant agents selected from List A.

The composition preferably comprises α-lipoic acid, or a salt thereof, and another one or more further anti-oxidant agents selected from List A. α-lipoic acid is also known as thioctic acid, and suitable salts include sodium lipoate.

The compositions preferably comprises carotenoids and another one or more further anti-oxidant agents selected from List A. Carotenoids may be used in the form of mixed carotenoids, and/or specific carotenoid species such as lutein, zeaxanthin or astaxanthin.

It is generally preferred that the composition should contain two anti-oxidant agents selected from List A, and should be free or substantially free of other anti-oxidant agents (apart from the ascorbic acid and tocopherol components of the composition, and optionally one or more metal ion anti-oxidant agents). In particular, it is preferred that the composition contains two anti-oxidant agents selected from List A and is free of other anti-oxidant agents set out in List A and free of other herbal extracts and vitamins (apart from the ascorbic acid and tocopherol components of the composition, and optionally one or more metal ion anti-oxidant agents).

Preferred compositions comprise carotenoids and one other anti-oxidant agent selected from List A, and are free of other anti-oxidant agents (apart from the ascorbic acid and tocopherol components of the composition, and optionally one or more metal ion anti-oxidant agents).

Preferred compositions comprise α-lipoic acid, or a salt thereof, and one other anti-oxidant agent selected from List A, and are free of other anti-oxidant agents (apart from the ascorbic acid and tocopherol components of the composition, and optionally one or more metal ion anti-oxidant agents).

Particularly preferred compositions comprise α-lipoic acid, or a salt thereof, and carotenoids, as well as ascorbic acid (or a salt, ester, glucoside, glucosamine and/or other derivative thereof) and tocopherol (or an ester and/or other derivative thereof).

The ascorbic acid component of the composition according to the invention may comprise ascorbic acid itself, but more preferably comprises a derivative of ascorbic acid. Examples of such derivatives include salts, eg sodium and calcium ascorbate, or esters with inorganic and organic acids, eg ascorbyl phosphate and ascorbyl palmitate. Most preferably, the ascorbic acid component of the composition is the ascorbic acid-derived product sold as “Ester-C” by Inter-Cal Nutraceuticals of Prescott, Ariz., USA. That product is understood to include calcium ascorbate, together with one or more derivatives of aldonic acids, particularly threonic acid, that are metabolites of ascorbic acid.

The tocopherol component of the composition according to the invention preferably comprises D-α-tocopherols, the naturally-occurring forms of Vitamin E. Tocopheryl salts may be used in various forms, with a variety of counterions. Tocopherol esters, eg D-α-tocopheryl acetate, may also be used.

For oral administration, the active ingredient may be put up in a variety of dosage forms. Preferably, the active ingredient will be formulated and administered as a solid dosage form, most commonly as a tablet or the like. Other solid dosage forms include capsules and lozenges, and formulation as a syrup (solution or suspension) may also be possible, as may other dosage forms.

For formulation in the presently preferred form, ie as a tablet, the active ingredient will generally be combined with various excipients in a manner which is known per se. In particular, the tablet will generally comprise one or more diluents or bulking agents. A lubricant may also be included to facilitate release of the formed tablets from the tabletting dies of a tablet forming machine.

Preferred materials for the diluent or bulking agents include polysaccharides and derivatives thereof, and saccharides.

Polysaccharides which may be used include starch, eg maize starch, cellulose, eg powdered cellulose and microcrystalline cellulose, water-insoluble modified starches, eg sodium carboxymethyl starch, water-insoluble cellulose derivatives, eg croscarmellose sodium (cross-linked sodium carboxymethyl cellulose), cross-linked polyvinylpyrrolidone and alginic acid.

Another preferred form of diluent is a saccharide. Suitable saccharides include, for example, sucrose, lactose, dextrose and sorbitol.

Particularly preferred diluents are microcrystalline cellulose, eg the products sold as Avicel PH-101 and Avicel PH-102 (Avicel is a Trade Mark) by the FMC Corporation of Philadelphia, Pa., USA, and lactose.

The lubricant may be, for example, stearic acid, a metallic stearate, a polyethylene glycol of molecular weight of 4,000 or more, or purified talc. The preferred lubricant is a metallic stearate, particularly magnesium stearate.

The tablet formulation may be prepared by methods that are familiar to those skilled in the art, eg processes involving wet or dry granulation or direct compression into a tablet without an intermediate, eg a wet or dry granulation, stage.

Preferred combinations of antioxidants in accordance with the present invention are combinations of
a) ascorbic acid, or a salt, ester, glucoside, glucosamine and/or other derivative thereof;
b) tocopherol, or an ester and/or other derivative thereof; and
c) one or, more preferably, two anti-oxidants selected from the following preferred list B.

Preferred List B:
carotenoids;
α-lipoic acid or a salt thereof;
hibiscus tea extract;
embilica extract;
rosehip extract;
elderflower extract; and
grape seed extract.

Preferred combinations of antioxidants in accordance with the invention include the following:
a) ascorbic acid, and its salts, esters, glucosides, glucosamines and/or other derivatives of ascorbic acid;
   tocopherol, or an ester and/or other derivative thereof;
   α-lipoic acid; and
   carotenoids.
b) ascorbic acid, and its salts, esters, glucosides, glucosamines and/or other derivatives of ascorbic acid;
   tocopherol, or an ester and/or other derivative thereof;
   rosehip extract; and
   elderflower extract.
c) ascorbic acid, and its salts, esters, glucosides, glucosamines and/or other derivatives of ascorbic acid;
   tocopherol, or an ester and/or other derivative thereof; and
   grape seed extract.
d) ascorbic acid, and its salts, esters, glucosides, glucosamines and/or other derivatives of ascorbic acid;
   tocopherol, or an ester and/or other derivative thereof; and
   α-lipoic acid or a salt thereof.
e) ascorbic acid, and its salts, esters, glucosides, glucosamines and/or other derivatives of ascorbic acid;
   tocopherol, or an ester and/or other derivative thereof; and
   lutein.
f) ascorbic acid, and its salts, esters, glucosides, glucosamines and/or other derivatives of ascorbic acid;
   tocopherol, or an ester and/or other derivative thereof; and
   hibiscus tea extract.
g) ascorbic acid, and its salts, esters, glucosides, glucosamines and/or other derivatives of ascorbic acid;
   tocopherol, or an ester and/or other derivative thereof; and
   embilica.

The compositions according to the invention preferably further comprise metal ion anti-oxidant agents. Suitable metal ions include zinc and, particularly, selenium ions. Such metal ions can be provided in various forms, with a variety of counter ions. Where selenium is used, as is preferred, the selenium is preferably provided as an organoselenium compound, eg a selenium amino acid or a mixture of selenium amino acids.

The antioxidants used in the invention are commercially available from numerous sources. For example:
ascorbic acid is preferably used in the form sold under the trade name “Ester C” by Inter-Cal Nutraceuticals of Prescott, Ariz., USA;
green tea (or camellia sinensis) extract is available under the trade name “Herbal Extract Green Tea 75% Solids” from Nichimen Europe;
rosehip extract is available from Flachsmann;
elderflower extract is available from Flachsmann;
grape seed extract is available from Chesham Chemicals Limited;
zinc may be used in the form of zinc gluconate available from Glucona BV of Veendam, The Netherlands;
α-lipoic acid is available from Astec Chemicals;
hibiscus tea extract is available from Chesham Chemicals Limited;
rooibus tea extract is available from Chesham Chemicals Limited;
tocopheryl acetate is available from Merck Speciality Chemicals;
embilica extract is available from Merck Speciality Chemicals;
carotenoids may be used in the commercially available form of “Mixed Carotenoids”; and
selenium may be used in the form a selenium yeast, chelate or salt, such as “Selenium Amino Acids”.

Effective doses of the combinations of anti-oxidants according to the invention may vary widely. However, typical daily dosages will be in the range of up to about 200 mg of each anti-oxidant. For the herbal extracts green tea, hibiscus tea, rooibus tea, embilica, rosehip, elderflower and grape seed, the daily dose may be in the range 1 mg to 20 mg. Similar levels of carotenoids and lutein may be employed. For ascorbic acid and tocopherol, and α-lipoic acid, the dose may be higher, eg 20 mg to 150 mg. For α-lipoic acid, the dose may be between 10 mg and 100 mg. For zinc and selenium ions, on the other hand, the daily dose will generally be orders of magnitude lower, eg 1 to 100 μg. The daily dose may be administered as a single dose, or as two or more divided doses. However, for greatest convenience, administration as a single daily dose is preferred.

The invention is illustrated, by way of example only, by the following Examples.

### EXAMPLE 1

Tablet for administration once per day

| Ingredient | Quantity (per tablet) | |
|---|---|---|
| Ester C | 141 | mg (equivalent to 100 mg |
| | | ascorbic acid) |
| Carotenoids | 6 | mg |
| Vitamin E | 100 | mg |
| α-Lipoic Acid | 50 | mg |
| Selenium | 25 | mcg |
| Magnesium Stearate | 11 | mg |
| Silicon Dioxide | 3 | mg |
| Calcium Sulphate | 80 | mg |
| Calcium Carbonate | 200 | mg |
| Microcrystalline Cellulose | 280 | mg |
| Sorbitol | 90 | mg |

### EXAMPLE 2

Tablet for administration twice per day

| Ingredient | Quantity (per tablet) | |
|---|---|---|
| Ester C | 70.5 | mg (equivalent to 50 mg |
| | | ascorbic acid) |
| Carotenoids | 3 | mg |
| Vitamin E | 50 | mg |
| α-Lipoic Acid | 25 | mg |
| Selenium | 25 | mcg |
| Magnesium Stearate | 10.5 | mg |
| Maize Starch | 42 | mg |
| Lactose | 150 | mg |

Other combinations of anti-oxidant agents that may be included in formulations similar to that of Example 1 (in place of the Ester C, Carotenoids, Vitamin E and α-lipoic acid) are set out in the following Examples 3 to 7:

### EXAMPLE 3

| Ingredient | Quantity (per tablet) | |
|---|---|---|
| Ester C | 141 | mg (equivalent to 100 mg |
| | | ascorbic acid) |
| Carotenoids | 6 | mg |
| Vitamin E | 100 | mg |
| Grapeseed extract | 12 | mg |

### EXAMPLE 4

| Ingredient | Quantity (per tablet) | |
|---|---|---|
| Ester C | 141 | mg (equivalent to 100 mg |
| | | ascorbic acid) |
| Carotenoids | 6 | mg |
| Vitamin E | 100 | mg |
| Rosehip extract | 8 | mg |
| Elderflower extract | 8 | mg |

### EXAMPLE 5

| Ingredient | Quantity (per tablet) | |
|---|---|---|
| Ester C | 141 | mg (equivalent to 100 mg |
| | | ascorbic acid) |
| Carotenoids | 6 | mg |
| Vitamin E | 100 | mg |
| Lutein | 6 | mg |

### EXAMPLE 6

| Ingredient | Quantity (per tablet) | |
|---|---|---|
| Ester C | 141 | mg (equivalent to 100 mg |
| | | ascorbic acid) |
| Carotenoids | 6 | mg |
| Vitamin E | 100 | mg |
| Hisbiscus Tea extract | up to 500 | mg |

### EXAMPLE 7

| Ingredient | Quantity (per tablet) | |
|---|---|---|
| Ester C | 141 | mg (equivalent to 100 mg |
| | | ascorbic acid) |
| Carotenoids | 6 | mg |
| Vitamin E | 100 | mg |
| Embilica extract | 30 | mg |

### EXAMPLE 8

Topical formulation

| Ingredient | % w/w |
|---|---|
| Vitamin C | 1.0 |
| Carotenoids | 0.06 |
| Vitamin E | 1.0 |
| α-Lipoic Acid | 0.5 |
| Selenium | 0.0002 |
| Carbopol | 0.2 |
| Sequestrene | 0.02 |
| Glycerin | 2.0 |
| Silicone | 1.5 |
| Cetearyl Alcohol | 2.16 |
| PEG-Stearate | 1.87 |
| Cetyl Alcohol | 1.8 |
| Liquid Paraffin | 10 |
| Glyceryl Stearate | 1.83 |
| Caustic Potash | 0.12 |
| Methyl Hydroxybenzoate | 0.2 |
| Propyl Hydroxybenzoate | 0.1 |
| Phenoxyethanol | 0.4 |
| Water | q.s. |

Further examples of topical formulations are the following, in which optional additional anti-oxidants selected from List A are indicated by square brackets:

### EXAMPLE 9

Oil-in-water emulsion

| lngredient | % w/w |
|---|---|
| Aqua | q.s. |
| Carbomer 940 | 0.35 |
| 1,3-Butylene glycol | 2 |
| Tetrasodium EDTA | 0.05 |
| Potassium Hydroxide | 0.06 |
| Polysorbate 80 | 1 |
| Paraffinum liquidum | 18 |
| Preservative | q.s |
| Ester C | 1.0 |
| Carotenoids | 0.45 |
| Vitamin E acetate | 0.25 |
| α-lipoic acid | 0.10 |
| zinc gluconate | 0.01 |
| Theobromo cacao | 0.5 |
| [Grape seed extract | 0.25] |

### EXAMPLE 10

Oil-in-water emulsion

| Ingredient | % w/w |
|---|---|
| Aqua | q.s. |
| Glycerin | 5 |
| Paraffinium liquidum | 5 |
| Dicaprylyl maleate | 3 |
| Petrolatum | 3 |
| Cetyl alcohol | 2 |
| Steareth-2 | 1.5 |
| Glyceryl stearate | 1.5 |
| Vitamin E | 0.8 |
| α-lipoic acid | 0.5 |
| Carotenoids | 0.25 |
| Vitamin C | 1.0 |
| Steareth-21 | 1 |
| Sodium citrate | 0.06 |
| Citric acid | 0.02 |
| Hydroxyethyl cellulose | 0.3 |
| Tetrasodium EDTA | 0.05 |
| Preservative | q.s |
| Parfum | q.s |
| [Hibiscus tea extract | 0.25] |

### EXAMPLE 11

Cold Cream

| Ingredient | % w/w |
|---|---|
| Aqua | q.s. |
| Magnesium aluminium silicate | 1 |
| Synthetic beeswax | 1.5 |
| Fatty acid ester | 1.5 |
| Paraffinum liquidum | 20 |
| Vitamin E | 0.8 |
| Vitamin C | 1.0 |
| α-lipoic acid | 0.25 |
| Carotenoids | 0.5 |
| Sorbitan monopalmitate | 3.5 |
| fatty acid ester | |
| Polysorbate 60 | 3.5 |
| Preservative | q.s |
| [Rosehip extract | 0.25] |

### EXAMPLE 12

Body Lotion

| Ingredient | % w/w |
|---|---|
| Aqua | q.s |
| Carbomer 940 | 0.3 |
| Sodium hydroxide | 0.028 |
| Glycerine | 5 |
| Glyceryl monostearate and polyoxyethylene | 4 |
| Stearate fatty acid ester | 2 |
| Paraffinum liquidum | 15 |
| Vitamin E | 0.8 |
| Vitamin C | 1.0 |
| α-lipoic acid | 0.25 |
| Carotenoids | 0.5 |
| Cholesterol | 5 |
| Oleyl alcohol | 2 |
| Tetrasodium EDTA | 0.05 |
| Preservative | q.s |
| [Rosehip extract | 0.25] |
| [Elderflower extract | 0.50] |

### EXAMPLE 13

Hair Conditioner

| Ingredient | % w/w |
|---|---|
| Aqua | q.s |
| Cetyl trimethyl ammonium chloride | 1.5 |
| Alumina | 0.5 |
| Petrolatum | 1.5 |
| Glyceryl stearate | 0.2 |
| Acetylated lanolin alcohol | 2 |
| Vitamin E | 0.8 |
| Vitamin C | 1.0 |
| α-lipoic acid | 0.5 |
| Carotenoids | 0.5 |
| Mineral oil (and) lanolin alcohol | 2 |
| Stearyl alcohol | 2.5 |
| Preservative | q.s |
| Parfum | q.s |
| [Rosehip extract | 0.25] |
| [Hibiscus tea extract | 0.50] |

### EXAMPLE 14

Fluid Foundation

| Ingredient | % w/w |
|---|---|
| Aqua | q.s |
| Carbomer 941 | 0.5 |
| Lanolin oil | 5 |
| Paraffinum liquidum | 3.5 |
| Stearyl alcohol (and) ceteareth-20 condensate | 3 |
| Triethanolamine | 0.5 |
| Ethanol | 26 |
| Vitamin E | 0.8 |
| Vitamin C | 1.0 |
| Alpha lipoic acid | 0.25 |
| Carotenoids | 0.5 |
| Tetrasodium EDTA | 0.02 |
| Pigments | q.s |
| Preservative | q.s |
| [Rosehip extract | 0.25] |
| [Grapeseed extract | 0.25] |

### EXAMPLE 15

Fluid Foundation

| Ingredient | % w/w |
|---|---|
| Aqua | q.s |
| 1,3-butylene glycol | 8 |
| Glycerin | 5 |
| Xanthan gum | 0.2 |
| Paraffinum liquidum | 4 |
| Cetearyl alcohol | 2 |
| PEG-20 stearate | 0.5 |
| Cetyl alcohol | 0.5 |
| Petrolatum | 1 |
| Vitamin E | 0.8 |
| Vitamin C | 1.0 |
| α-lipoic acid | 0.25 |
| Carotenoids | 0.5 |
| Zinc gluconate | 0.10 |
| Theobromo cacao | 1.5 |
| BHT | 0.02 |
| Dimethicone | 5 |
| Tetrasodium EDTA | 0.05 |
| Preservative | q.s |
| Pigments | q.s |
| [Elderflower extract | 0.25] |

### EXAMPLE 16

Sun Lotion

| Ingredient | % w/w |
|---|---|
| Aqua | q.s |
| Ethanol | 10 |
| Octyldodecanol | 8 |
| Glycerin | 5 |
| Octyl methoxycinnamate | 2 |
| Octocrylene | 1 |
| Hydrogenated coco-glycerides | 3 |
| Butyl methoxydibenzoylmethane | 2 |
| Drometrizole trisiloxane (Mexoryl XL) | 3 |
| Terephthalylidene dicamphor sulfonic | 3 |
| acid (Mexoryl SX) | |
| 4-methylbenzylidene camphor | 1 |
| C12-15 alcohols benzoate | 1 |
| Coco-glucoside | 1 |
| Vitamin E | 0.8 |
| Vitamin C | 1.0 |
| α-lipoic acid | 0.25 |
| Carotenoids | 0.5 |
| Theobromo cacao | 0.5 |
| Preservative | q.s |
| Parfum | q.s |
| [Rosehip extract | 0.25] |
| [Hibiscus tea extract | 0.50] |

In all of the above Examples, the anti-oxidants selected from List A may be replaced by other such anti-oxidants, or further anti-oxidants selected from List A (or indeed other anti-oxidants) may be included in addition to those named.

The following experiments were carried out to verify the properties of combinations of anti-oxidants in accordance with the invention.

1. In Vitro Studies

1.1 Aim

The aim was to determine whether protection from UV-induced DNA damage can be provided by the tested combinations of anti-oxidants.

1.2 Materials and Methods

Human dermal fibroblasts were collected from two subjects, one aged 33 years and the other aged 55 years.

1.3 “COMET” Assay

The so-called COMET assay is used to measure damage to DNA after irradiation. After being exposed to a test substance, cells are embedded on agarose on a glass plate and connected to a power source. DNA in the cells will migrate towards the positive pole. DNA fragments resulting from damage to the DNA molecules will migrate faster in the electric field than intact DNA. If the DNA is appropriately stained, the fragments are visible as “comet tails” under fluorescence microscopy conditions. A quantification of DNA damage can be derived from the ratio of comet tail and head (nucleus) intensities and sizes.

In the present case, the assay was employed to measure the degree of DNA damage induced by UV light in human dermal fibroblast cells in culture. The cells were incubated with the test material for 18 hours, and then irradiated with a standard level of UV light (780 mJ/cm of simulated solar light from a Xe arc lamp). The irradiated samples were then subjected to COMET assay both immediately after irradiation and two hours after irradiation. The samples were electrophoresed for 30 minutes, and the results were processed using an Olympus BX30 fluorescence microscope with image analysis software supplied by Perceptive Instruments of Haverhill, Suffolk, UK.

1.4 Test Materials

Test materials were prepared as follows:

a) Comparative samples

| | |
|---|---|
| Control | No added anti-oxidants; not subjected to UV exposure |
| Standard | No added anti-oxidants; subjected to UV exposure |

b) Test samples (all subjected to irradiation)

| | |
|---|---|
| A | Selenium amino acid + carotenoids + Vitamin E - |
| | referred to as “Stock A” |
| A + gt | Stock A + green tea extract |
| A + zn | Stock A + zinc gluconate |
| A + R + E + Ec | Stock A + rosehip extract + elderflower extract + |
| | “Ester C” |
| A + Ec + G | Stock A + “Ester C” + grape seed extract |
| A + Ec + Al | Stock A + “Ester C” + α-lipoic acid |

It will be noted that the final three combinations listed above fall within the scope of the present invention.

1.5 Results

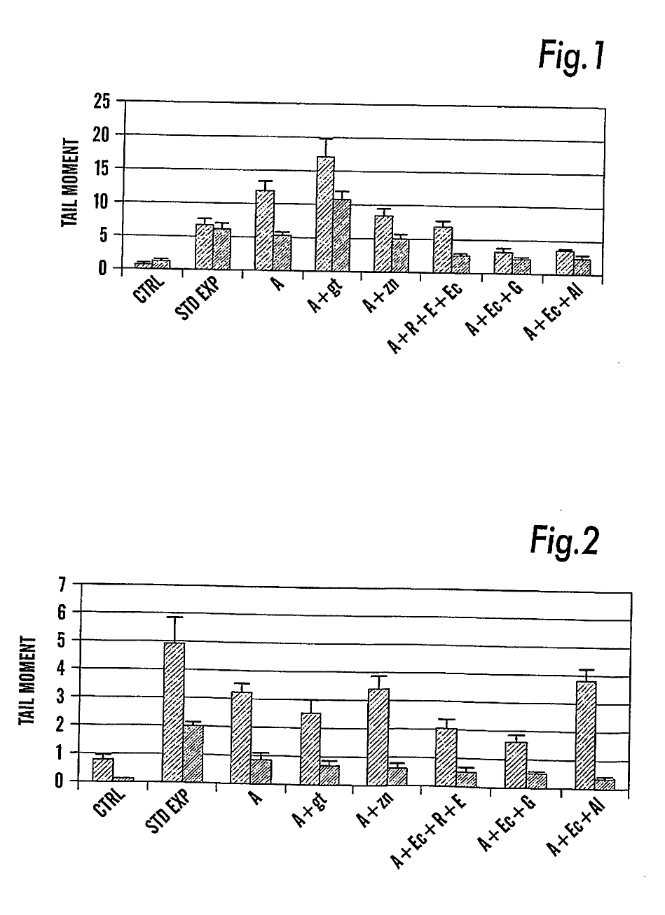

The “Tail moment” measured for each sample immediately after, and two hours after, irradiation is shown in FIG. 1 for the samples obtained from the 33-year old subject, and in FIG. 2 for the cells obtained from the 55-year old subject. For each sample, the result indicated by the left-hand bar is that obtained immediately after irradiation, and the result obtained two hours after irradiation is indicated by the right-hand bar.

1.6 Conclusions

For the “standard”, DNA damage is induced by exposure to UV radiation. That damage is repaired to a certain degree over the course of two hours, but still remains considerably higher than the degree of DNA damage indicated for the control sample that was not exposed to UV radiation.

For the cells from the 33-year old subject, pre-treatment with certain anti-oxidants (“Stock A”, alone or with the addition of green tea or zinc) actually caused an increase in tail moment immediately post-irradiation. For the A+gt sample the tail moment remained elevated 2 hours post-irradiation.

For the combinations according to the invention, however, there was no significant increase in tail moment immediately post-irradiation (and in two cases there was a significant reduction), and the tail moment 2 hours post-irradiation was significantly reduced in all cases.

For the cells obtained from the 55-year old subject, the tail moments were all reduced relative to the standard immediately post-irradiation, and were the lowest of all tested combinations 2 hours after irradiation.

2. In Vivo Studies

2.1 Aim

A study was designed to test the efficacy of a tablet formulation containing the same combination of anti-oxidants as Example 1 above, compared with a placebo formulation comprising no anti-oxidants.

2.2 Methods

60 volunteers were recruited, of whom 54 completed the trial. The volunteers, who were mainly female, with an average age of 43 years, were divided into two groups (placebo and test). The volunteers took one tablet per day for a period of three months. Measurements were taken as described below prior to the start of the trial, and at monthly intervals until the trial was completed.

Volunteers presented at monthly intervals and skin samples were taken by tape stripping from sun-protected sites on their bodies (lower back). Two samples were taken each time from each volunteer and were stored in a freezer until all samples had been collected. Once all samples had been collected, one of each pair of samples was assigned to a “control” group and the other to an “exposed” group. The exposed group samples were irradiated under a solar simulator for a standard period and subsequently stored in the freezer until the trial was completed and all samples had been collected.

After completion of the trial, the samples were all removed from the freezer and the lipids on the tape strippings were extracted in methanol, centrifuged, and aliquots of the supernatant were taken and assayed for the presence of lipid hydroperoxides using the Kamiya Biotech K-Assay. This assay is calorimetric and each experimental run is calibrated to a blank and a standard of known concentration (cumene hydroperoxide).

2.3 Results

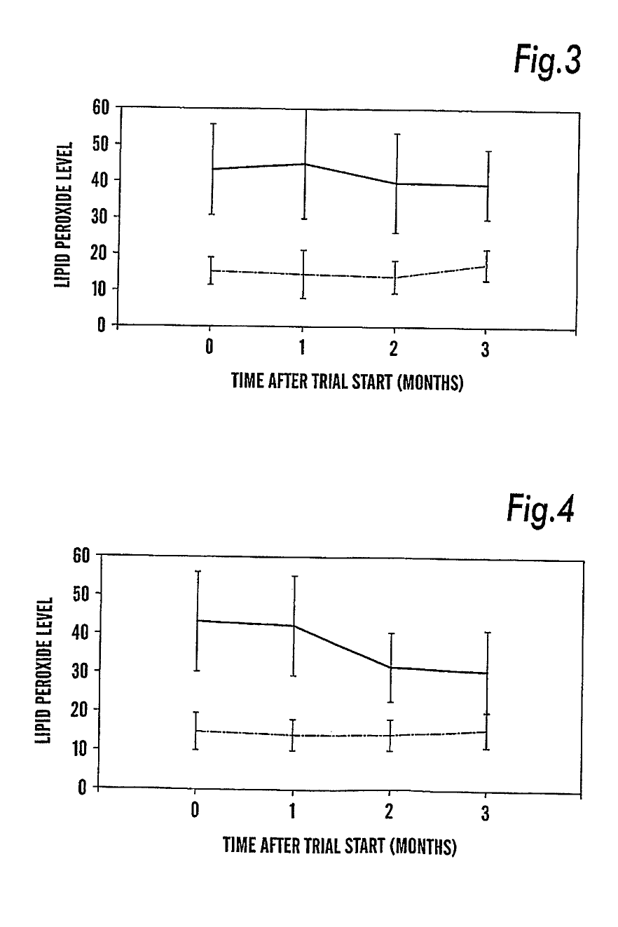

FIG. 3 shows the average lipid peroxide levels in irradiated (solid line) and non-irradiated (broken line) samples for volunteers taking the placebo. FIG. 4 shows corresponding data for volunteers taking the anti-oxidant formulation.

2.4 Conclusions

FIG. 3 shows that the placebo had no effect on the level of lipid peroxide either in unexposed or irradiated skin samples. This demonstrates the expected effect in that placebo was inactive and irradiation induces an approximate three-fold increase in background levels of lipid peroxides. This data also supports a steady background level of lipid peroxidation, that would imply that any change seen in volunteers taking the anti-oxidant formulation would indeed be due to the treatment and not to an environmental artefact.

For the volunteers who took the anti-oxidant formulation, FIG. 4 shows no change over time for the unexposed skin samples. However, for the irradiated skin samples, there is a significant decrease in lipid peroxidation level between the initial state and 2 months after the trial start (p=0.000315) and 3 months after the trial start (p=0.000237).

2.5 Follow-Up Study

Five months after completion of the original study, further samples were taken from 17 of the original volunteers who had taken the anti-oxidant formulation and from 5 volunteers who had taken the placebo. These samples were analysed in the same manner as the original samples.

It was found that the lipid peroxide levels for these volunteers had returned to the same or similar levels as were measured for the same volunteers at the beginning of the original study.

1. A composition for the prevention or inhibition of free radical-induced effects on the skin, which composition comprises
a) ascorbic acid, or a salt, ester, glucoside, glucosamine and/or other derivative thereof;
b) tocopherol, or an ester and/or other derivative thereof; and
c) at least two further anti-oxidant agents selected from the group consisting of:
carotenoids;
α-lipoic acid and salts thereof;
green tea extract;
hibiscus (Sudanese) tea extract;
rooibus tea extract;
embilica extract;
rosehip extract;
elderflower extract; and
grape seed extract;
the composition optionally comprising one or more metal ion antioxidant agents, and wherein the composition is free or substantially free of other antioxidant agents.

2. A composition as claimed in claim 1, which comprises two or more of said further anti-oxidant agents.

3. A composition as claimed in claim 1, which comprises α-lipoic acid, or a salt thereof, and another one or more of said further anti-oxidant agents.

4. A composition as claimed in claim 1, which comprises carotenoids and another one or more of said further anti-oxidant agents.

5. A composition as claimed in claim 1, which comprises
a) ascorbic acid, or a salt, ester, glucoside, glucosamine and/or other derivative thereof;
b) tocopherol, or an ester and/or other derivative thereof; and
c) two of said further anti-oxidant agents;
the composition being free or substantially free of other anti-oxidant agents.

6. A composition as claimed in claim 5, which comprises
a) ascorbic acid, or a salt, ester, glucoside, glucosamine and/or other derivative thereof;
b) tocopherol, or an ester and/or other derivative thereof; and
c) two, and only two, of said further anti-oxidant agents;
the composition being free of other herbal extracts and vitamins.

7. A composition as claimed in claim 1, which comprises
a) ascorbic acid, or a salt, ester, glucoside, glucosamine and/or other derivative thereof;
b) tocopherol, or an ester and/or other derivative thereof;
c) α-lipoic acid, or a salt thereof; and
d) carotenoids.

8. A composition as claimed in claim 1, which comprises a metal ion anti-oxidant agent.

9. A composition as claimed in claim 8, wherein the metal ion anti-oxidant agent comprises selenium ions.

10. A composition as claimed in claim 1, wherein the composition is in a form suitable for oral administration.

11. A composition as claimed in claim 10, which is a solid dosage form.

12. A composition as claimed in claim 10, which is in the form of a tablet.

13. A composition as claimed in claim 1, which comprises from 1 mg to 20 mg each of anti-oxidants selected from carotenoids, green tea extract, hibiscus tea extract, rooibus tea extract, embilica extract, rosehip extract, elderflower extract and grape seed extract.

14. A composition as claimed in claim 1, which comprises between 20 mg and 150 mg of ascorbic acid, or a salt, ester, glucoside, glucosamine and/or other derivative thereof.

15. A composition as claimed in claim 1, which comprises between 20 mg and 150 mg of tocopherol, or an ester and/or other derivative thereof.

16. A composition as claimed in claim 1, which comprises between 10 mg and 100 mg of α-lipoic acid or a salt thereof.

17. A composition as claimed in claim 1, which is in a form suitable for topical application to the skin.

18. A method for the prevention or inhibition of free radical-induced effects on the skin, which method comprises the administration of a composition as claimed in claim 1.

19. A method as claimed in claim 18, in which the composition is administered orally.

20. A method as claimed in claim 19, in which the composition is administered as a solid dosage form.

21. A method as claimed in claim 18, in which the composition is administered as a tablet.

22. The use of
a) ascorbic acid, or a salt, ester, glucoside, glucosamine and/or other derivative thereof;
b) tocopherol, or an ester and/or other derivative thereof; and
c) at least two further anti-oxidant agents selected from the group consisting of:
carotenoids;
α-lipoic acid and salts thereof;
green tea extract;
hibiscus (Sudanese) tea extract;
rooibus tea extract;
embilica extract;
rosehip extract;
elderflower extract; and
grape seed extract;
the composition optionally comprising one or more metal ion anti-oxidant agents in the manufacture of a composition for the prevention or inhibition of free radical-induced effects on the skin, wherein the composition is free or substantially free of other antioxidant agents.
